(19) United States
(12) Patent Application Publication
(10) Pub. No.: US 2017/0298321 A1
(43) Pub. Date: Oct. 19, 2017

(54) METHODS FOR IMPROVING PLANT EMBRYO QUALITY AND GERMINATION

(71) Applicants: Pramod K. Gupta, Federal Way, WA (US); Mayumi Ikeda, Tacoma, WA (US)

(72) Inventors: Pramod K. Gupta, Federal Way, WA (US); Mayumi Ikeda, Tacoma, WA (US)

(21) Appl. No.: 15/336,303

(22) Filed: Oct. 27, 2016

Related U.S. Application Data
(60) Provisional application No. 62/265,567, filed on Dec. 10, 2015.

(51) Int. Cl. C12N 5/04

(52) U.S. Cl. CPC C12N 5/04; C12N 2500/34; C12N 2500/50

(57) ABSTRACT

Provided is a method of producing plant embryos having improved embryo quality or germination frequency comparing to embryos developed by conventional methods. The method entails the steps of (a) incubating plant embryogenic suspensor mass (ESM) in, or on, a standard development medium supplied with glucose for a first incubation period to develop immature somatic embryos; (b) mass transferring the immature somatic embryos to a modified development medium near or at cotyledon stage; and (c) incubating the immature somatic embryos in, or on, the modified development medium for a second incubation period to develop mature somatic embryos. The modified development medium contains reduced concentration of glucose or is glucose-free and the osmolality of the modified development medium is adjusted to compensate for the loss of osmolality due to reduction or removal of glucose.

### CROSS REFERENCE AND RELATED APPLICATION

This application claims priority to U.S. Patent Application No. 62/265,567, filed Dec. 10, 2015, which is incorporated herein by reference in its entirety. To the extent the foregoing application and/or any other materials incorporated herein by reference conflict with the present disclosure, the present disclosure controls.

### FIELD OF THE TECHNOLOGY

The present technology relates to improving plant embryo quality such as germination frequency.

### BACKGROUND

The demand for high quality plants, especially trees such as coniferous trees, for forestry and for making wood products continues to increase. One proposed solution to the problem of providing an adequate supply of plants is to identify individual plants that possess desirable characteristics, such as a rapid rate of growth and improved wood quality, and to reproduce these plants having the desirable traits. Thus, modern silviculture often requires the planting of large numbers of genetically identical plants that have been selected to have advantageous properties. Production of new plants by sexual reproduction, which yields botanic seeds, is usually not feasible. Asexual propagation via the culturing of somatic or zygotic embryos has been shown to yield large numbers of genetically identical embryos, each having the capacity to develop into a normal plant.

Somatic cloning is the process of creating genetically identical plants from plant somatic tissue other than the male and female gametes. In one approach to somatic cloning, plant tissue is cultured in an initiation medium which includes hormones such as auxins and/or cytokinins to initiate formation of embryogenic tissue, such as embryogenic suspensor masses, that are capable of developing into somatic embryos. Embryogenic suspensor mass (ESM) has the appearance of a whitish translucent mucilaginous mass and contains early stage embryos. The embryogenic tissue is further cultured in a multiplication medium that promotes multiplication and mass production of the embryogenic tissue. The embryogenic tissue is then cultured in a development medium that promotes development and maturation of cotyledonary somatic embryos which can, for example, be placed on a germination medium to produce germinants, and subsequently transferred to soil for further growth; or alternatively, placed within manufactured seeds and sown in the soil where they germinate to produce seedlings. Manufactured seeds are described, for example, in U.S. Pat. Nos. 5,564,224; 5,687,504; 5,701,699; and 6,119,395.

Although some plants can be reproduced by somatic cloning, somatic embryo germination sometimes is very low compared to zygotic embryo germination. Somatic embryo quality contributes to germination frequency.

There is a continuing need for methods for producing high quality plant somatic embryos to promote maturation and germination of somatic embryos.

### SUMMARY

In one aspect, a method of producing plant embryos having improved embryo quality is provided. The method comprises reducing or removing glucose in the modified development medium while maintaining or increasing osmolality during late stage of embryo development. The method entails the steps: (a) incubating plant embryogenic suspensor mass (ESM) in, or on, a development medium comprising glucose for a first incubation period to develop immature somatic embryos; (b) mass transferring the immature somatic embryos to a modified development medium near or at cotyledon stage; and (c) incubating the immature somatic embryos in, or on, the modified development medium for a second incubation period to develop mature somatic embryos, wherein the mature somatic embryos have improved embryo quality comparing to the mature somatic embryos developed without transferring to or incubating in, or on, the modified development medium. The osmolality of the modified development medium is maintained at from about 250 mM/Kg to about 500 mM/Kg during the second incubation period. The improved embryos exhibit increased germination frequency. In certain embodiments, the plant is a tree, such as a conifer.

### BRIEF DESCRIPTION OF THE DRAWINGS

FIG. 1 shows loblolly pine embryos at the time of mass transfers. Genotype A (top row) was transferred at two weeks (left), three weeks (middle) and four weeks (right) post plating. Genotype B (bottom row) was transferred at three weeks (left), four weeks (middle), and five weeks (right) post plating.

FIG. 2 shows mean yield by treatment for loblolly pine genotypes A and B, determined at 12 weeks after plating. Error bars represent standard deviation. Treatment 1: embryos were not moved and stratification continued on standard development medium (BM 4); Treatment 2: embryos were mass moved to high PEG/glucose-free medium (BM 11) at pre-dome/dome stage (genotype A moved two weeks after plating, and genotype B moved three weeks after plating); Treatment 3: embryos were mass moved to high PEG/glucose-free medium (BM 11) at pre-cotyledon stage (genotype A moved three weeks after plating, and genotype B moved four weeks after plating); and Treatment 4: embryos were mass moved to high PEG/glucose-free medium (BM 11) at cotyledon stage (genotype A moved four weeks after plating, and genotype B moved five weeks after plating).

FIGS. 3A and 3B show mean percent germination by treatment for loblolly pine genotype A (FIG. 3A) and genotype B (FIG. 3B). Error bars represent standard deviation. Treatment 1: embryos were not moved and stratification continued on standard development medium (BM 4); Treatment 2: embryos were mass moved to high PEG/glucose-free medium (BM 11) at pre-dome/dome stage (genotype A moved two weeks after plating, and genotype B moved three weeks after plating); Treatment 3: embryos were mass moved to high PEG/glucose-free medium (BM 11) at pre-cotyledon stage (genotype A moved three weeks after plating, and genotype B moved four weeks after plating); and Treatment 4: embryos were mass moved to high PEG/glucose-free medium (BM 11) at cotyledon stage (genotype A moved four weeks after plating, and genotype B moved five weeks after plating).

FIGS. 4A and 4B show mean yield per plate (FIG. 4A) and mean yield per ml settled cell volume (SCV) (FIG. 4B) by treatment for loblolly pine genotype A. Treatment 1: embryos were not moved and continued on standard development medium (BM 4); Treatment 2: embryos were mass moved to standard development medium (BM 4) at dome/pre-cotyledon stage (approximately three to four weeks after plating); and Treatment 3: embryos were mass moved to high PEG/glucose-free medium (BM 11) at dome/pre-cotyledon stage (approximately three to four weeks after plating).

FIG. 5 shows mean percent germination by treatment for loblolly pine genotype A. Treatment 1: embryos were not moved and continued on standard development medium (BM 4); Treatment 2: embryos were mass moved to standard development medium (BM 4) at dome/pre-cotyledon stage (approximately three to four weeks after plating); and Treatment 3: embryos were mass moved to high PEG/glucose-free medium (BM 11) at dome/pre-cotyledon stage (approximately three to four weeks after plating).

FIG. 6 shows percent germination (normalized to control) by treatment in a scale-up test of mass transfer of loblolly pine to high PEG/glucose-free medium. Treatment A (control): embryos developed on standard development medium (BM 4) were not moved and then stratified in BM 6 medium; and Treatment B: embryos developed on standard development medium (BM 4) were moved to high PEG/glucose-free medium (BM 11) at dome/cotyledon stage and then stratified in BM 6 medium.

FIG. 7 shows 6 weeks germination of loblolly pine Genotype 3 and Genotype 4 for treatments A and B. Treatment A (control): embryos developed on standard development medium (BM 4) were not moved and then stratified in BM 6 medium; and Treatment B: embryos developed on standard development medium (BM 4) were moved to high PEG/glucose-free medium (BM 11) at dome/cotyledon stage and then stratified in BM 6 medium.

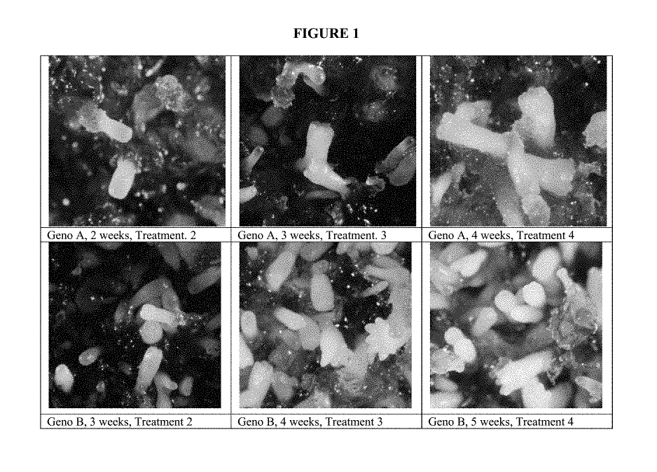

### DETAILED DESCRIPTION

Unless specifically defined herein, all terms used herein have the same meaning as they would to one skilled in the art of the disclosed technology.

As used herein, the term “embryogenic suspensor mass” (ESM) refers to early stage embryos in the process of multiplication by budding and cleavage.

As used herein, the term “embryogenic tissue” refers to an aggregate of tens to hundreds of embryogenic cells that form an embryogenic suspensor mass.

As used herein, the term “somatic embryo” refers to an embryo produced by culturing embryogenic tissue by standard methods under laboratory conditions in which the cells comprising the tissue are separated from one another and urged to develop into minute complete embryos.

The different developmental stages of embryos are determined by the embryos' external morphology. As used herein, the term “cotyledonary embryo” refers to an embryo that possesses one or more cotyledons. Cotyledonary embryos have a well-defined elongated bipolar structure with latent meristem with cotyledonary primordia at one end and a potential radicle at the opposite end. The cotyledonary structure frequently appears as a small “crown” at one end of the embryo. The term “pre-cotyledonary embryo” refers to an embryo that does not yet have cotyledons. Similarly, dome stage means that an embryo has developed an apical dome, which is the part of the apical meristem interior to the leaf primordia. The term “pre-dome embryo” refers to an embryo that does not yet have an apical dome.

As used herein, the phrase “plant embryos having improved embryo quality” means that the embryos have improved germination frequency and/or improved morphology compared to conventional embryo quality and germination methods, including more symmetrical shape, substantially straight hypocotyl region, an opaque or white color. Preferably, the plant embryos having improved embryo quality produced by the methods disclosed herein have qualities similar to zygotic embryos. The germinants developed from these improved embryos have increased root length and improved hypocotyl and epicotyl comparing to the germinants developed from embryos produced by conventional methods. The term “germination frequency” refers to the number, proportion, percentage or fraction of germinants in a particular population of somatic embryos.

As used herein, the term “modified development medium” means that a standard development medium is modified to reduce or remove the glucose content. For example, for a standard development medium containing about 1% glucose, the modified development medium contains less than 1%, less than 0.9%, less than 0.8%, less than 0.7%, less than 0.6%, less than 0.5%, less than 0.4%, less than 0.3%, less than 0.2%, or less than 0.1% glucose. In certain embodiments, the modified development medium is glucose-free. Further, one or more osmoticants are added to the modified development medium to compensate for the decrease of osmolality due to reduced glucose content.

The somatic embryogenesis process is a process to develop plant embryos in vitro. This process has significant uses in forestry and in making wood products. Methods for producing plant somatic embryos are known in the art and have been previously described (see, e.g., U.S. Pat. Nos. 4,957,866; 5,034,326; 5,036,007; 5,041,382; 5,236,841; 5,294,549; 5,482,857; 5,563,061; and 5,821,126). Generally, the somatic embryogenesis process includes the steps of (1) initiating formation of embryogenic tissue (“initiation” or “induction”), such as embryogenic suspensor mass (ESM), which is a white mucilaginous mass that includes early stage embryos having a long, thin-walled suspensor associated with a small head with dense cytoplasm and large nuclei; (2) multiplying and mass producing the embryogenic tissue (“multiplication” or “maintenance”); (3) developing and forming mature cotyledonary somatic embryos (“development”); and (4) post-development processes such as stratification, singulation, and conditioning over water (COW), and then germination or placement into manufactured seeds.

During embryo development, carbohydrate or sugar sources are related to good quality embryo development. The standard development medium described in this disclosure contains two sugars as a carbohydrate source, glucose (which is monosaccharide) and maltose (which is disaccharide). Both sugars promote proliferation and embryo development during the development stage.

As described herein, it has been unexpectedly discovered that reducing or removing glucose from the standard development medium during late stage embryo development produces embryos having improved quality as compared to the embryos developed in or on the standard development medium without reducing or removing glucose. Although the presence of monosaccharide such as glucose is important during embryo multiplication and early development stages, reducing or removing glucose from the development medium during late stage of embryo development, for example, near or at cotyledon stage, significantly improves embryo quality, particularly germination frequency.

The present technology provides a method of producing plant embryos having improved embryo quality. In several embodiments, methods in accordance with the present technology include: (a) incubating plant embryogenic suspensor mass (ESM) in, or on, a standard development medium supplied with glucose for a first incubation period to develop immature somatic embryos; (b) mass transferring the immature somatic embryos to a modified development medium near or at cotyledon stage; and (c) incubating the immature somatic embryos in, or on, the modified development medium for a second incubation period to develop mature somatic embryos, wherein the mature somatic embryos have improved embryo quality comparing to mature somatic embryos developed without transferring to or incubating in, or on, the modified development medium. In one embodiment, the osmolality of the modified development medium is maintained at from about 250 mM/Kg to about 500 mM/Kg during the second incubation period. The improved embryos exhibit increased germination frequency. In certain embodiments, the plant is conifer.

It is desirable to include a carbohydrate source such as glucose or maltose in the development medium during the first incubation period to promote the growth and multiplication of conifer embryogenic tissue, such as embryonal suspensor mass. The first incubation period is about 2 weeks, about 3 weeks, about 4 weeks, about 5 weeks, about 6 weeks, about 7 weeks, or about 8 weeks, depending on the conifer species. The immature somatic embryos are mass transferred to a modified development medium at or near cotyledon stage, for example, at pre-dome/dome stage, at pre-cotyledon stage, or at cotyledon stage, determined by the external morphology of the embryos. Mass transfer to the modified development medium can occur at multiple time points during late development stage of embryos to achieve similar improved results.

After mass transfer, the embryos are incubated in the modified development medium for a second incubation period to develop mature somatic embryos. The second incubation period is about 4 weeks, about 5 weeks, about 6 weeks, about 7 weeks, or about 8 weeks, such that the total incubation period is from about 8 weeks to about 14 weeks.

In some embodiments, the modified development medium is completely depleted of glucose or glucose-free. In other embodiments, the modified development medium contains glucose at a reduced concentration comparing to the standard development medium. For example, the glucose concentration in the modified development medium is less than 1% (w/v), less than 0.9% (w/v), less than 0.8% (w/v), less than 0.7% (w/v), less than 0.6% (w/v), less than 0.5% (w/v), less than 0.4% (w/v), less than 0.3% (w/v), less than 0.2% (w/v), or less than 0.1% (w/v) glucose.

Due to reduction or removal of glucose, the osmolality of the modified development medium during the second incubation decreases, which may cause a negative effect on embryo development. One or more osmoticants are added to the modified development medium such that the osmolality of the modified development medium is maintained within a range from about 250 mM/Kg to about 500 mM/Kg during the second incubation period. In certain embodiments, the osmolality is from about 250 mM/Kg to about 450 mM/Kg, from about 250 mM to about 400 mM/Kg, from about 250 mM to about 350 mM/Kg, from about 250 mM to about 300 mM/Kg, from about 300 mM/Kg to about 500 mM/Kg, from 350 mM/Kg to about 500 mM/Kg, from about 400 mM/Kg to about 500 mM/Kg, or from about 350 mM/Kg to about 450 mM/Kg.

Preferably, the osmoticants are non-penetrating osmoticants that do not penetrate into plant cells and do not cause detrimental effects to plant cells. Examples of osmoticants that can be added to adjust osmolality in the development medium include, but are not limited to, PEG having a molecular weight from 1000 to 8000. The PEG concentration in the modified development medium can be from about 10% to about 15% (w/v). It is within the purview of one ordinary skill in the art to select PEG having a particular molecular weight and to adjust the concentration of PEG to achieve the desired osmolality in the modified development medium.

In certain embodiments, the plants disclosed herein are trees, including conifers such as pines and firs. For example, the plant is a conifer such as loblolly pine or Douglas-fir. In certain embodiments, the standard development medium or the modified development medium used in this technology is a liquid medium, a semi-solid medium, or a solid medium.

After the development period, the somatic embryos can optionally be transferred to a maturation medium, and then subjected to post-development steps such as singulation, stratification, germination, placement into manufactured seeds, and transferring to soil for further growth and development.

In several embodiments, the improved embryos developed by the method of the present technology exhibit increased germination frequency and/or improved morphology compared to conventional methods, including more symmetrical shape, substantially straight hypocotyl region, an opaque or white color. Preferably, the plant embryos having improved embryo quality produced by the methods disclosed herein have qualities similar to zygotic embryos. Additionally, the germinants developed from the improved embryos have increased root length and improved hypocotyl and epicotyl compared to the germinants developed from embryos produced by conventional methods.

As shown in Examples 2-4, the embryos developed by transferring to a modified, glucose-free medium during the late development stage have significantly increased germination frequency, although the yield of the embryos is not significantly increased by medium transfer.

The following examples are provided for the purpose of illustration and are not intended to limit the disclosure in any way.

### EXAMPLES

### Example 1

This example shows the compositions of the media used in the examples that follow. The modified solid development medium (BM 11) removes glucose and increases the content of PEG comparing to the standard development medium (BM 4).

TABLE 1

| | Media | | | | | |
|---|---|---|---|---|---|---|
| | BM 2 | BM 3 | BM 4 | BM 11 | BM 6 | BM 7 |
| Description | Maintenance | Rinse | Solid | Modified | Cold (about | Germination |
| | Liquid | | Development | Solid | 1° C. to | |
| | | | | Development | about 8° C.) | |

Salts (mg/L)

| | | | | | | |
|---|---|---|---|---|---|---|
| NH₄NO₃ | 150 | 150 | 150 | 150 | 150 | 206.25 |
| KNO₃ | 909.9 | 909.9 | 909.9 | 909.9 | 909.9 | 1170 |
| Ca(NO₃)2—4H₂O | 236.15 | 236.15 | 236.15 | 236.15 | 236.15 | |
| MgSO₄—7H₂O | 246.5 | 246.5 | 246.5 | 246.5 | 246.5 | 185 |
| Mg(NO₃)₂—6H₂O | 256.5 | 256.5 | 256.5 | 256.5 | 256.5 | |
| MgCl₂—6H₂O | 50 | 50 | 50 | 50 | 50 | |
| KH₂PO₄ | 136 | 136 | 136 | 136 | 136 | 85 |
| CaCl₂—2H₂O | 50 | 50 | 50 | 50 | 50 | 220 |
| KI | 4.15 | 4.15 | 4.15 | 4.15 | 4.15 | 0.415 |
| H₃BO₃ | 15.5 | 15.5 | 15.5 | 15.5 | 15.5 | 3.1 |
| MnSO₄•H₂O | 10.5 | 10.5 | 10.5 | 10.5 | 10.5 | 8.45 |
| ZnSO₄—7H₂O | 14.4 | 14.4 | 14.4 | 14.4 | 14.4 | 4.3 |
| Na₂MoO₄—2H₂O | 0.125 | 0.125 | 0.125 | 0.125 | 0.125 | 0.125 |
| CuSO₄—5H₂O | 0.125 | 0.125 | 0.125 | 0.125 | 0.125 | 0.0125 |
| CoCl₂—6H₂O | 0.125 | 0.125 | 0.125 | 0.125 | 0.125 | 0.0125 |
| FeSO₄—7H₂O | 27.87 | 27.87 | 27.87 | 27.87 | 13.93 | 13.93 |
| Na₂EDTA | 37.26 | 37.26 | 37.26 | 37.26 | 18.63 | 18.63 |

Vitamins/Amino Acids (mg/L)

| | | | | | | |
|---|---|---|---|---|---|---|
| Nicotinic Acid | 0.5 | 0.5 | 0.5 | 0.5 | 0.5 | 0.5 |
| Pyridoxine HCl | 0.5 | 0.5 | 0.5 | 0.5 | 0.5 | 0.5 |
| Thiamine HCl | 1 | 1 | 1 | 1 | 1 | 1 |
| Glycine | 2 | 2 | 2 | 2 | 2 | 2 |
| L-proline | | 100 | 100 | 100 | 100 | |
| L-asparagine | | 100 | 100 | 100 | 100 | |
| L-arginine | | 50 | 50 | 50 | 50 | |
| L-alanine | | 20 | 20 | 20 | 20 | |
| L-serine | | 20 | 20 | 20 | 20 | |
| PEG 8000 mw | | | 10000 | 13000 | | |

Sugar/Agar mg/L

| | | | | | | |
|---|---|---|---|---|---|---|
| Myo-Inositol | 200 | 100 | 100 | 100 | 100 | 100 |
| Casein hydrolysate | 500 | 500 | 500 | 500 | 500 | |
| L-glutamine | 1000 | 1000 | 1000 | 1000 | 1000 | |
| Sucrose | | | | | 25000 | 20000 |
| Maltose | 30000 | 25000 | 25000 | 20000 | | |
| Glucose | | | 10000 | | | |
| GELRITE* | | | 2500 | 2500 | 2500 | |
| TC Agar | | | | | | 8000 |
| Activated carbon | | | 1000 | 1000 | 1000 | 2500 |

Hormones mg/L

| | | | |
|---|---|---|---|
| ABA* | | 25 | 25 |
| 2,4-D* | 1.1 | | |
| BAP* | 0.1 | | |
| Kinetin* | 0.1 | | |

GELRITE: gellan gum (an agar substitute);
ABA: abscisic acid;
2,4-D: 2,4-dichlorophenoxyacetic acid;
BAP: 6-benzylaminopurine;
Kinetin: 6-furfurylaminopurine

### Example 2

This example shows the effects of removing glucose in combination with adjusting osmolality by increasing PEG during embryo late development stage on germination.

Loblolly pine embryogenic suspensor mass (ESM) from two different genotypes, Genotype A and Genotype B, were obtained from WAVE bags which were maintained in liquid medium BM 2 (as described in Example 1) at 20° C. Samples of the settled cells were rated, and 50 mls of cells were transferred to a cyto-stir and an equivalent amount of rinse medium BM 3 (as described in Example 1) was added to achieve a 1:1 ratio. Adding rinse medium helped in spreading cells onto membrane. The cells were plated according to standard procedure using vacuum pressure system (VPS) units with Decotex 2″×2″ membranes, as described in U.S. Pat. No. 8,925,245, the content of which is incorporated by reference. Each plating consisted of 54 plates of each genotype.

Post-development, the loblolly pine somatic embryos were subjected to stratification (20 ml/plate), followed by conditioning over water (COW) for two weeks. Subsequently, the somatic embryos were hand transferred for germination using the method as described in U.S. Pat. No. 9,090,872, the content of which is incorporated by reference herein in its entirety. Germination was assessed at 6 weeks, in 100 ml of BM 7 medium (as described in Example 1).

The approximate timeline for the experiment was as follows: embryos were developed for about 8-14 weeks after plating and followed by 4 weeks of stratification. After conditioning over water for an additional 2 weeks, the embryos were germinated. The germination was assessed after 6 weeks.

Assessment of plates occurred at 12 weeks of development. A cursory assessment was at 8 weeks after plating to note any differences in rate of development between treatments. Final assessment included notes about embryo size, length, shape, color, texture, cotyledon formation, amount and condition of ESM, and plate to plate or within-plate variation.

Plate yield was determined at the point of singulation prior to conditioning over water (COW), and yields were assessed on half of a given plate as long as there were 25 or more embryos on the half plate. If there were less than 25 embryos on half the plate, the entire plate was assessed. Plate yields were tracked through to germination so that germinants/ml could be assessed. Six plates of 25 embryos per plate were laid as the embryo numbers allowed for germination. Germination assessments consisted of root length and epicotyl (longest) measurements of embryos.

The embryos were subjected to the following selection criteria: symmetrical embryos without obvious defects that had four or more cotyledons without any fused cots or cots sprouting from the center were chosen. Embryos had all three parts, cotyledons, hypocotyl, and radicle regions. Sizes of the embryos varied. A slight curve to the hypocotyl region was acceptable. Embryos with split radicle regions were not selected. Embryos were opaque and colors were shades of white, yellow or green. Translucent embryos or vitrified green embryos were not selected. Only the embryos having good quality determined by these selection criteria were germinated.

In this example, the ESM was multiplied/developed on solid development medium (BM 4) and then some was mass transferred to a modified solid development medium (BM 11) in which glucose was removed and PEG was increased at different times. The treatments are detailed in Table 2 below.

TABLE 2

| | | | Myo- | | PEG | | Plating | | |
|---|---|---|---|---|---|---|---|---|---|
| | | Osmo | inositol | Maltose | (% | Glucose | volume | | |
| Treatment | Media | (mM/Kg) | (mg/l) | (% w/v) | w/v) | (% w/v) | (mL) | Mass Transfer | Stratification |
| 1 | BM 4 | 353 | 100 | 2.5 | 10 | 1 | 0.5 | No | continue on |
| | | | | | | | | | BM4 |
| 2 | BM 4 | 353 | 100 | 2.5 | 10 | 1 | 0.5 | to BM 11 at | continue on |
| | | | | | | | | pre-dome/dome | BM11 |
| 3 | BM 4 | 353 | 100 | 2.5 | 10 | 1 | 0.5 | to BM 11 at | continue on |
| | | | | | | | | pre-cotyledon | BM11 |
| 4 | BM 4 | 353 | 100 | 2.5 | 10 | 1 | 0.5 | to BM 11 at | continue on |
| | | | | | | | | cotyledon | BM11 |

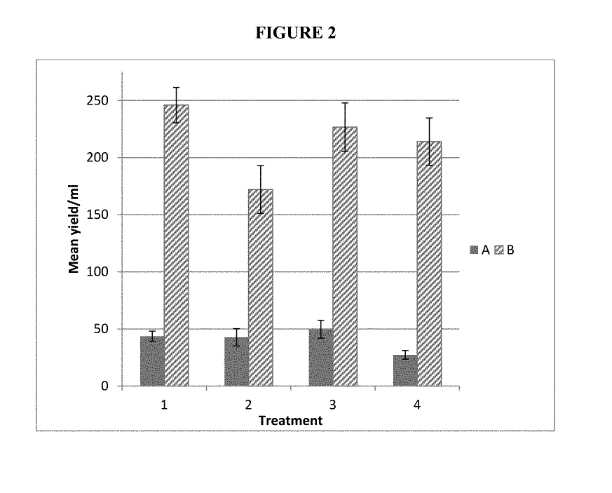

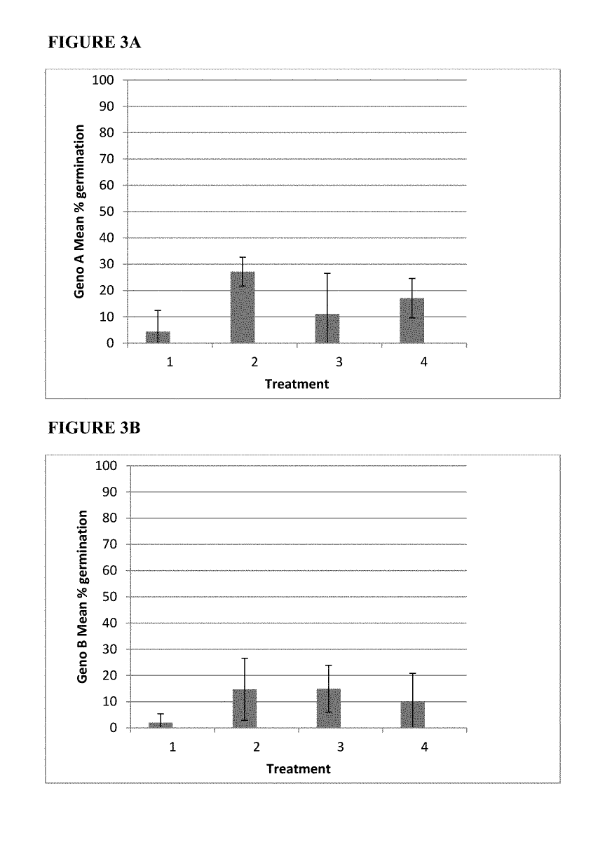

As shown in FIG. 2, transferring to a modified medium did not result in any statistically significant improvement in the mean yield for both genotypes. By contrast, as shown in FIGS. 3A and 3B, germination frequency of the good quality embryos was significantly improved for both genotypes when the ESM was cultured on the standard development medium (BM 4) and then mass transferred to the modified development medium removing glucose and increasing PEG (BM 11) during late development stage. The PEG content in BM 11 was increased to compensate for the loss of osmolality due to removal of glucose. Removing glucose during the late development stage, whether it occurred at pre-dome/dome (treatment 2), at pre-cotyledon (treatment 3), or at cotyledon (treatment 4), did not affect the results of improving germination frequency. Comparing to good quality embryos developed without medium transfer, the germination frequency of Genotype A good quality embryos increased from about 5% to about 11%-28%; the germination frequency of Genotype B increased from about 2% to about 10%-15%.

### Example 3

Similar to Example 2, this example shows the effects of removing glucose in combination of adjusting osmolality by increasing PEG during embryo late development stage on germination.

Loblolly pine embryogenic suspensor mass (ESM) from Genotype A was obtained from WAVE bags which were maintained in liquid medium BM 2 (as described in Example 1) at 20° C. Samples of the settled cells were rated, and 75 mls of cells were transferred to a cyto-stir and an equivalent amount of rinse medium BM 3 (as described in Example 1) was added to achieve a 1:1 ratio. Adding rinse medium helped in spreading cells onto membrane. The cells were plated according to the standard procedure using vacuum pressure system (VPS) units with Decotex 2″×2″ membranes, as described in U.S. Pat. No. 8,925,245, the content of which is incorporated by reference. Each plating consisted of 54 plates of each genotype. The mass transfer occurred when the embryos reached the dome/pre-cotyledon stage determined by visual assessment of the external morphology, approximately three to four weeks after plating. All plates were transferred to stratification media BM 6 (as described in Example 1) prior to being placed into the cold.

The post-development processes were performed in the same way as described in Example 2, with the exception that the stratification was carried out according to standard procedure in medium BM 6 at a cold temperature, e.g., from 1° C. to 8° C. The plate assessment was carried out in the same way as described in Example 2, with the exception that a cursory assessment was done from 5 to 8 weeks rather than at 8 weeks after plating. The selection criteria for embryos were the same as described in Example 2.

In this example, the ESM was multiplied/developed on the standard development medium (BM 4) and then some was mass transferred to BM 4 (Treatment 2) and some to the modified development medium (BM 11) in which glucose was removed and PEG was increased (Treatment 3). The treatments are detailed in Table 3 below.

TABLE 3

| | | Myo- | | PEG | | Plating | |
|---|---|---|---|---|---|---|---|
| Treat- | | inositol | Maltose | (% | Glucose | volume | Mass |
| ment | Media | (mg/l) | (% w/v) | w/v) | (% w/v) | (mL) | Transfer |
| 1 | BM 4 | 100 | 2.5 | 10 | 1 | 0.5 | No |
| 2 | BM 4 | 100 | 2.5 | 10 | 1 | 0.5 | BM 4 |
| 3 | BM 4 | 100 | 2.5 | 10 | 1 | 0.5 | BM 11 |

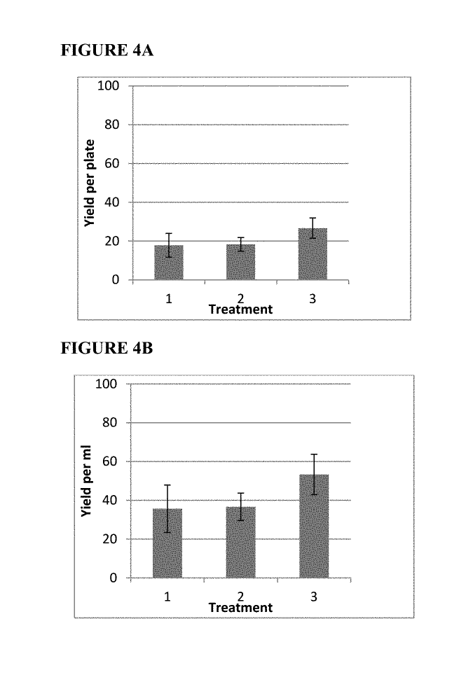

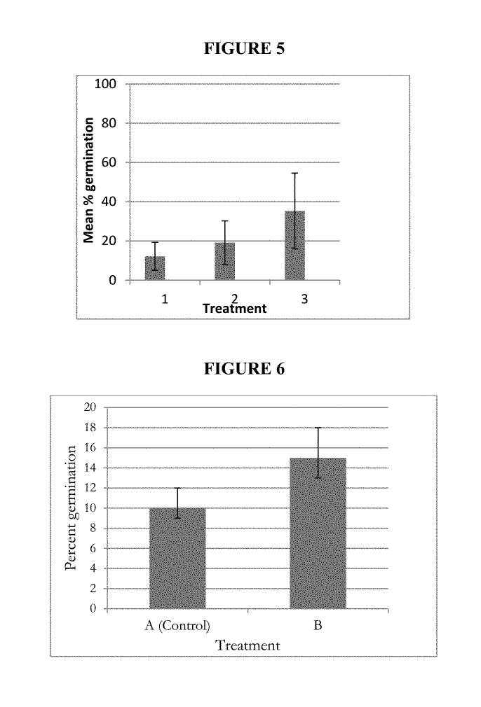

As shown in FIG. 4, transferring to the standard medium BM4 did not increase the yield, while transferring to the modified medium BM 11 slightly increased the yield. As shown in FIG. 5, Treatment 3 which was good quality embryos from settled cell volume (SCV) plated on the standard medium and then transferred to the modified, glucose-free medium had the best yield per ml as well as germination per ml. The germination frequency of good quality embryos for Treatment 3 increased from about 12% (Treatment 1, control) to about 36% (Treatment 3). Transferring to the standard medium BM 4 also increased the germination frequency of good quality embryos to some extent, from about 12% (Treatment 1, control) to about 19% (Treatment 2), probably due to the presence of a fresh medium. This result is consistent with the results obtained in Example 2.

### Example 4

This example shows the effects of removing glucose in combination of adjusting osmolality by increasing PEG during embryo late development stage on germination at a mass production scale.

In view of the consistent improvement in germination achieved in the foregoing examples, a scale-up test of mass transfer of loblolly pine to a high PEG/glucose-free development medium was performed.

Four genotypes were plated and the experiment was performed in a similar way as described in Examples 2 and 3, with the exception that the embryos were automatically transferred onto germination medium BM 7 in ⅓ Cambro boxes. The ESM was mass transferred at the same stage as described above to 400 ml transfer medium in ⅓ Cambro boxes.

The post-development processes were modified accordingly. The loblolly pine somatic embryos were subjected to stratification in BM 6 (as described in Example 1) in 200 per ⅓ Cambro box, followed by either spray separation and singulation or conditioning over water (COW) for two weeks. Spray separated embryos were kept in COW for a week; therefore, no imbibitions for spray separated embryos were required. Subsequently, the somatic embryos were transferred for germination using the method as described in U.S. Pat. No. 9,090,872, the content of which is incorporated by reference. Germination was assessed at 6 weeks, in 400 ml of BM 7 medium (as described in Example 1).

In this example, the ESM was multiplied/developed on the standard development medium (BM 4) and then some was mass transferred to the modified development medium (BM 11) in which glucose was removed and PEG was increased. The treatments are detailed in Table 4 below.

TABLE 4

| Treatment | Media | Mass Transfer | Stratification |
|---|---|---|---|
| A | BM 4 | No | BM 6 |
| B | BM 4 | BM 11 | BM 6 |

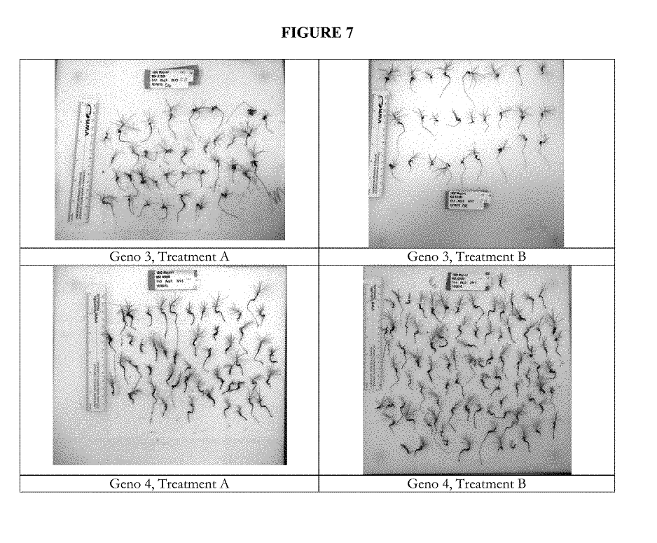

A scaled-up mass transfer of embryos during the late development stage to the high PEG/glucose-free medium significantly increased germination. As shown in FIG. 7, the representative genotypes, Genotype 3 and Genotype 4 exhibit improved germinant morphology, e.g., increased root length, substantially straight hypocotyl, etc. in Treatment B group comparing to Treatment A group (control).

We claim:

1. A method of producing plant embryos having improved embryo quality and/or germination frequency comprising:
(a) incubating plant embryogenic suspensor mass (ESM) in, or on, a standard development medium comprising glucose for a first incubation period to develop immature somatic embryos;
(b) mass transferring the immature somatic embryos to a modified development medium near or at cotyledon stage; and
(c) incubating the immature somatic embryos in, or on, the modified development medium for a second incubation period to develop mature somatic embryos,
wherein the modified development medium is glucose-free or contains glucose at a concentration of less than 1%, and
wherein the mature somatic embryos have improved embryo quality and/or germination frequency comparing to mature somatic embryos developed without transferring to or incubating in, or on, the modified development medium.

2. The method of claim 1, wherein the plant is a conifer.

3. The method of claim 1, wherein the mass transfer occurs at predome/dome stage, at pre-cotyledon stage, or at cotyledon stage.

4. The method of claim 1, wherein the osmolality of the modified development medium during the second incubation period is between about 250 mM/Kg and about 500 mM/Kg.

5. The method of claim 1, wherein one or more osmoticants are added to the modified development medium during the second incubation period.

6. The method of claim 5, wherein the osmoticant is a non-penetrating osmoticant.

7. The method of claim 5, wherein the osmoticant is PEG having a molecular weight between 1000 and 8000.
